(19) United States
(12) Patent Application Publication
(10) Pub. No.: US 2007/0184462 A1
(43) Pub. Date: Aug. 9, 2007

(54) Apparatus and method for solid-phase kinetic analysis of templated elongation reactions

(76) Inventors: Zachary Burton, Haslett, MI (US); Yuri Nedialkov, East Lansing, MI (US); Xueqian Gong, East Lansing, MI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 11/524,114

(22) Filed: Sep. 20, 2006

Related U.S. Application Data
(60) Provisional application No. 60/719,627, filed on Sep. 21, 2005.

(51) Int. Cl. C12Q 1/68; C12M 3/00

(52) U.S. Cl. 435/6; 435/287.2

(57) ABSTRACT

Disclosed is an elongation reaction system that includes a membrane compatible with binding an elongation complex and an elongation complex compatible with binding the membrane. The elongation complex includes the biological template (e.g., DNA or RNA), a polymerizing agent (e.g., RNA polymerase, DNA polymerase, or a ribosome), and a primer transcript or polypeptide. Further, disclosed is an apparatus and method for solid-phase kinetic analysis of templated elongation reactions.

### CROSS-REFERENCE TO RELATED APPLICATION

This application claims the benefit under 35 U.S.C. §119(e) of U.S. Provisional Application No. 60/719,627, entitled APPARATUS AND METHOD FOR SOLID-PHASE KINETIC ANALYSIS OF TEMPLATED ELONGATION REACTIONS, filed on Sep. 21, 2005, the entire disclosure of which is hereby incorporated herein by reference.

### FIELD OF THE INVENTION

This invention relates to apparatuses and methods for analyzing biological templated replication, transcription, and translation reactions.

### BACKGROUND OF THE INVENTION

A significant portion of the genomes of living organisms are dedicated to transcription and replication: RNA and DNA synthesis. The elongation phase of the transcription cycle is regulated by elongation factors, chromatin, DNA-binding proteins and specific DNA and RNA sequences. The fidelity of RNA synthesis is very high, yet the precise mechanisms governing fidelity of NMP incorporation are just beginning to emerge, primarily from kinetic studies of human RNA polymerase II (RNAP II) elongation. Gong, X. Q. et al., Alpha-Amanitin Blocks Translocation by Human RNA Polymerase II, J Biol. Chem. 279, 27422-27427 (2004); Nedialkov, Y. A. et al., NTP-Driven Translocation by Human RNA Polymerase II, J Biol. Chem. 278, 18303-18312 (2003a); and Zhang, C., et al., Transcription Factors IIF and IIS and Nucleoside Triphosphate Substrates as Dynamic Probes of the Human RNA Polymerase II Mechanism, J Mol. Biol. 342, 1085-1099 (2004). The fidelity of DNAP elongation, furthermore, is an issue of huge importance, because DNAP fidelity regulates mutation rates, for instance, in genesis of cancer. Kaguni, L. S., DNA Polymerase Gamma, The Mitochondrial Replicase, Annu. Rev. Biochem. 73, 293-320. Replicative DNAPs generally possess very high fidelity in order to copy the genome accurately and minimize accumulation of mutations. Some DNAPs, however, have evolved to be more error-prone. These enzymes enhance the rate of mutations or replicate severely damaged DNA, when more accurate mechanisms of replication become impractical. Thus, understanding gene regulation and the fidelity and mechanism of essential biological polymerization reactions are significant and important undertakings.

Transient state kinetics analyses of RNAPs have significant advantages over traditional steady-state analyses. Gong et al. (2004); Nedialkov et al. (2003a); Nedialkov, Y. A. et al., Transient State Kinetics of RNA Polymerase II Elongation, Methods Enzymol. 371, 252-262 (2003b); and Zhang (2004). Transient state analysis allows the synchronous rates of synthesis of multiple specific phosphodiester bonds to be tracked with millisecond precision. Measurements in the millisecond phase are important because this is the time scale in which most relevant protein motions and conformational changes occur. Detailed information is gained about the mechanism or formation of a single bond, about escape from a transcriptional stall, and about the process of synthesis of multiple bonds. In transient state analysis, the correlation between measured rates and elementary reaction steps is apparent, because rates can be well-resolved and easily distinguished. Because single, specific catalytic events are monitored, transient state analysis provides much more reliable insight into reaction mechanism than traditional steady-state analysis, in which elementary reaction steps are blurred because multiple events are observed simultaneously, rather than discreetly, as in transient state measurements. Johnson, K. A., Rapid Quench Kinetic Analysis of Polymerases, Adenosinetriphosphatases, and Enzyme Intermediates, Methods Enzymol. 249, 38-61 (1995). A detailed, quantitative, and testable model for an enzymatic mechanism provides the necessary background for determining how a reaction is regulated and how small molecule effectors, such as drugs, affect essential cellular processes. Coupling atomic structures, kinetic analysis, mutation analysis, and molecular dynamics, RNAP and DNAP mechanisms and regulations, can be understood in detail. Because RNAPs and DNAPs are targets for drug design (i.e., antibiotics and antivirals) and drug toxicity testing (i.e., human mitochondrial DNAP γ), detailed and quantitative mechanistic studies of these essential enzymes take on added significance.

For human RNAP II and E. coli RNAP, transient state analysis has been richly informative. Human RNAP II has been analyzed by the inventors through formation of multiple specific bonds, giving insight into escape from a transcriptional stall and into normal processive transitions between one bond and the next. Gong et al. (2004), Nedialkov et al. (2003a), Nedialkov (2003b), Zhang (2004), and Zhang, C. et al., Combinatorial Control of Human RNA Polymerase II (RNAP II) Pausing and Transcript Cleavage by Transcription Factor IIF, Hepatitis δ (delta) Antigen and Stimulatory Factor II, J Biol. Chem. 278, 50101-50111 (2003). Reported experiments with E. coli RNAP have been limited to single bond addition studies. Foster, J. E. et al., Allosteric Binding of Nucleoside Triphosphates to RNA Polymerase Regulates Transcription Elongation, Cell 106, 243-252 (2001); and Holmes, S. F. et al., Downstream DNA Sequence Effects on Transcription Elongation. Allosteric Binding of Nucleoside Triphosphates Facilitates Translocation Via a Ratchet Motion, J Biol. Chem. 278, 35597-35608 (2003). However, experiments should be done monitoring the addition of multiple bonds, as demonstrated for RNAP II.

Compared to single-molecule elongation studies of E. coli RNAP, which currently can only approach single-bond resolution, transient state kinetic studies have distinct advantages in revealing details of the RNAP II mechanism. Neuman, K. C. et al., Ubiquitous Transcriptional Pausing Is Independent of RNA Polymerase Backtracking, Cell 115, 437-447 (2003); and Shaevitz, J. W. et al., Backtracking by Single RNA Polymerase Molecules Observed at Near-Base-Pair Resolution, Nature 426, 684-687 (2003).

The inventors have previously applied transient state kinetic methods to study elongation by human RNAP II and its regulation by transcription factor IIF (TFIIF), transcription factor IIS (TFIIS), hepatitis δ (delta) antigen, and α-amanitin. Gong et al. (2004), Nedialkov et al. (2003a), Zhang (2004a), and Zhang (2003). Using transient state kinetic methods, the inventors also have studied yeast RNAP II, with or without yeast TFIIF, and with or without yeast TFIIS. The yeast system is amenable to construction, tagging, and isolation of mutant proteins (RNAP II and elongation factors) so both the mechanism and regulation of RNA synthesis can be examined in detail. The inventors also have applied transient state kinetic analysis to study E. coli RNAP. Other laboratories, as well, have been conducting such studies. Foster et al. (2001) and Holmes et al. (2003). The reasons to develop the E. coli system are: 1) to screen for novel antibiotics; and 2) to study mutant forms of multi-subunit RNAPs. Others also have applied transient state kinetic methods to study human DNAP γ, 2-subunit DNAP for mitochondrial (mt) DNA replication. Kaguni, L. S., DNA Polymerase γ (gamma), the Mitochondrial Replicase, Annu. Rev. Biochem. 73, 293-320 (2004). There is also increased recognition that antiviral and anti-tumor drugs frequently affect mitochondrial metabolism, and in particular, the activity of mitochondrial DNAP γ. Johnson, A. A. et al., Toxicity of Antiviral Nucleoside Analogs and the Human Mitochondrial DNA Polymerase, J Biol. Chem. 276, 40847-40857 (2001).

Currently, liquid-phase transient state kinetic analysis is performed using rapid pipetting devices, such as the KinTek Rapid Chemical Quench-Flow (RQF-3) instrument (KinTek Corporation, Austin, Tex.). Such transient state kinetic analysis allows tracking of elongation rates through formation of individual bonds with millisecond precision. However, use of such rapid pipetting devices is cumbersome and slow. The rapid pipetting device, also known as “liquid-phase” or “mobile-phase” kinetic analysis device, forces an elongation complex in buffer solution into contact with nucleoside triphosphates (NTPs) under pressure for milliseconds to elongate a nascent transcript. Specifically, a DNA template carrying a biotin group at the 5′ end is linked and immobilized on a streptavadin agarose bead, e.g., Promega MagneSphere Beads (Madison, Wis.). Transcription is initiated by adding a selected RNAP and forming a nascent RNA transcript. The immobilized DNA template, RNAP enzyme, and nascent RNA transcript are collectively referred to as an “elongation complex” or “EC”. The KinTek RQF-3 instrument permits elongation reactions to be started and stopped (quenched) within 0.002 seconds. A solution of HCl or ethylene diamine-tetra-acetic acid (EDTA) is used to quench the elongation reaction and/or release the elongated transcript from the EC.

FIG. 1 shows a schematic drawing of a known rapid chemical quench-flow mixing instrument. The instrument shown has three syringes 1, 2, and 3, under the control of a drive motor (not shown). Typically, elongation complexes in transcription buffer are loaded into syringe 1 and nucleotide triphosphate (NTP) substrates in transcription buffer, at twice their working concentration, are loaded into syringe 2. Initiation of a computerized reaction program activates the drive motor to push the solution from both syringes 1 and 2 into mixing chamber 4. The reaction program then loads quench solution from syringe 3 to mixing chamber 5 to stop or quench the elongation reaction. Alternatively, using a 3-syringe instrument, after a pulse of a first NTP solution (e.g., ATP) from syringe 2 and mixing of the first NTP solution with elongation complex solution from syringe 1 at mixing chamber 4, a second NTP solution (e.g., GTP) is added to the reaction from syringe 3 into mixing chamber 5. In this design, quenching later occurs when the mixed solution moves to collection tube 6, which includes a quench solution, such as EDTA. A known 4-syringe rapid chemical quench-flow instrument (not shown) can be utilized to add one more reaction.

Using known designs, the time needed to add an NTP to a primer transcript, in the presence of various substrates, can be measured. Such substrates include transcription/elongation factors; drugs and antibiotics, e.g., Microcin J25, CBR 703 Series (A new class of bacterial RNA polymerase inhibitor affects nucleotide addition. 2003. Artsimovitch, I., Chu, C., Lynch, A. S., and Landick, R. Science 302: 650-654), and rifampicin; and toxic molecules, such as α-amanitin. Generally, if the NTP is quickly bound to the primer RNA in low concentration of the NTP, then the NTP has a high affinity for the RNA transcript. Using the known apparatus, the elongated RNA transcript is analyzed by gel electrophoresis (e.g., by fluorescently labeling the transcript). The kinetic analysis apparatus allows elongation to be stalled in the elongation complex at defined positions with millisecond timing. In sum, in the known rapid pipetting device, the elongation complexes encounter reagents “in-flow”, that is, the elongation complexes in solution flow into the reaction chamber and then flow out. Hydrochloric acid (HCl) is used to release the elongated RNA transcript from the RNAP, after which elongation of the transcript is studied by gel electrophoresis.

There are, however, several deficiencies with known liquid-phase rapid quench-flow instruments. Namely, liquid-phase analysis of templated elongation kinetics: 1) provides inconsistent results, because the number of elongation complexes immobilized on beads in solution varies from experiment to experiment; 2) the number of permitted reactions is limited by a three or four syringe configuration. Moreover, the addition of syringes would be ineffective in that, with additional syringes, millisecond timing necessary for transient state kinetic analysis would be difficult, if not impossible, to maintain. Accordingly, the known liquid-phase rapid pipetting devices are inflexible in accommodating complicated experimental designs that require multiple reagents or different elongation complexes. Finally, using the known liquid-phase rapid pipetting devices, the limiting step in analyzing the functional dynamics of RNAPs and DNAPs is the large amount of time required for collection of high-quality kinetic data sets. That is, the liquid-phase instrument is not high-throughput.

### SUMMARY OF THE INVENTION

In one embodiment, the present invention includes an elongation reaction system including a membrane compatible with binding an elongation complex and an elongation complex compatible with binding the membrane. The elongation complex includes a biological template (e.g., DNA or RNA), a polymerizing agent (e.g., RNA polymerase, DNA polymerase, or a ribosome), and a primer transcript or polypeptide. In this embodiment of the invention, the membrane is porous and the polymerizing agent is fully processive.

Another aspect of the present invention is to provide an apparatus for millisecond kinetic analysis of a templated elongation reaction including, a membrane compatible with anchoring an elongation complex, a reaction chamber adapted to receive the membrane, at least two reagents, and a feed-line interconnecting the reagents and the reaction chamber. In this aspect, the apparatus includes elongation complexes immobilized to the membrane; and reagents may include NTPs, transcription factors, toxins, drugs, and quench solution. The apparatus also includes a computer for controlling the flow of reagents to the reaction chamber, as well as one or more valves for controlling delivery of the reagent to the feed-line. In another embodiment, the apparatus of the present invention is high-throughput and may include any of the following: an instrument for printing the template on the membrane, an apparatus for moving the membrane into the reaction chamber, a fraction collector, and an apparatus for gel electrophoresis or other high throughput detection (i.e., capillary electrophoresis).

In another embodiment, the present invention includes a method for millisecond kinetic analysis of a templated elongation reaction. The method includes providing an apparatus for millisecond kinetic analysis including a membrane compatible with binding the elongation complex, a reaction chamber, at least two reagents, and a feed-line interconnecting the reagents and the reaction chamber; binding to the membrane an elongation complex, the elongation complex including a template, a polymerizing agent, and a primer; moving the membrane into the reaction chamber; and independently flowing at least two reagents over the elongation complex to permit elongation of the primer and quenching of the elongation.

These and other features, advantages and objects of the present invention will be further understood and appreciated by those skilled in the art by reference to the following specification, claims and appended drawings.

### BRIEF DESCRIPTION OF THE DRAWINGS

FIG. 1 is a schematic drawing of a prior art rapid pipetting device;

FIG. 2 is a diagram of an elongation complex bound to a membrane;

FIG. 3 is a top plan view of an array of elongation complexes immobilized on membranes;

FIG. 4 is a side view of one embodiment of the apparatus of the present invention;

FIG. 5 is an enlarged side view of the reaction chamber shown in FIG. 3;

FIG. 6 is a perspective view of another embodiment of the apparatus of the present invention; and

FIG. 7 is a top plan view of a wheel of sample cells.

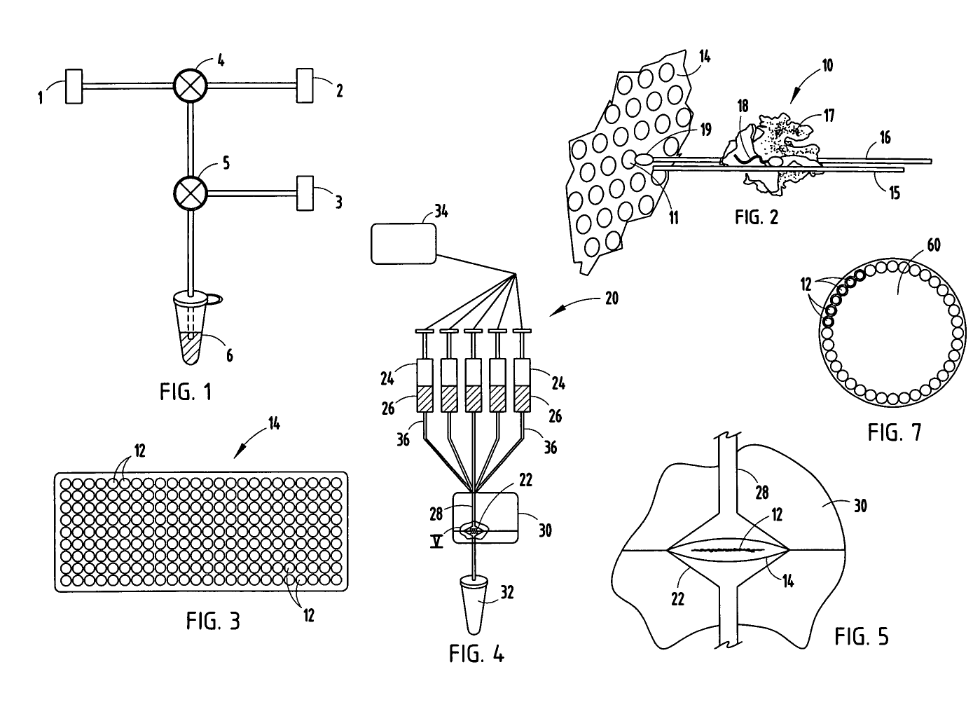

### DETAILED DESCRIPTION OF THE INVENTION

The present invention may be understood more readily by reference to the following detailed description of the specific embodiments and the drawings.

As used in the application, “a” can mean one or more, depending on the context with which it is used.

As used in the application, the term “oligonucleotide” refers to primers, probes, and oligomer fragments to be detected.

As used in the application, the term “primer” refers to an oligonucleotide or a polypeptide, whether natural or synthetic, which is capable of acting as a point of initiation, when placed under conditions in which synthesis of a primer extension product, which in the instance of an oligonucleotide is complementary to a template nucleic acid strand is induced. For example, in the presence of nucleotides and a “polymerizing agent” (such as RNA polymerase, DNA polymerase, or a ribosome) and at a suitable temperature and pH, the primer acts as a point of initiation or synthesis by the polymerizing agent.

In general, the present invention relates to “solid-phase” kinetic analysis of a templated reaction, i.e., a preparation of elongation complexes immobilized or anchored in a reaction chamber, and an apparatus and method for analyzing solid-phase templated elongation complexes in the reaction chamber. The invention allows for multiple reagent additions with millisecond start-stop times, providing an unprecedented view of RNAP, DNAP, and ribosomal catalytic mechanisms and their regulation by elongation factors and small molecule effectors. Using the present invention, kinetic analyses will be the basis for drug development and testing, experimental designs will become more flexible (multiple reagent additions, instead of single reagent additions), and analyses can be high-throughput (1,000 reactions per day) with improved precision and reproducibility. Solid-phase analysis according to the present invention also will allow acquisition of much more comprehensive data sets. As such, many more confirmatory experiments can be performed using each experimental system.

The immobilized elongation complex can be a replication complex, transcription complex, or translation complex. Each type of elongation complex includes a DNA or RNA template, a polymerizing agent (DNAP, RNAP, or a ribosome), and a primer (a nascent DNA or RNA transcript complementary to the DNA template, or a nascent polypeptide encoded by the ribosome). The DNA or RNA template is a known sequence that has been stalled at a defined template position. The primer can be radioactively labeled (e.g., with ³²P) or fluorescently labeled for identification and study after elongation. The preferred embodiment of the present invention includes promoter-less initiation of elongation of the EC. Such promoter-less initiation of elongation, as compared to promoter-specific initiation of the elongation reaction, is less expensive and more efficient.

During the elongation phase of the transcription cycle, most RNAPs are fully processive polymerizing agents, an important property utilized by the present invention. Specifically, a fully processive RNAP can be immobilized or anchored, along with its nascent RNA product (or primer) and DNA template in an elongation complex (EC), and an elongation reaction can be maintained in solid-phase, flowing reagents past the immobile elongation complex, to prime, pulse, chase, and stop the reaction. A fully processive polymerizing agent is fully committed to the template and elongation product. For example, throughout reagent additions, a fully processive RNA polymerase remains associated with the DNA template and the RNA primer/transcript. Using a fully processive polymerizing agent, one million-fold less material is needed for an elongation reaction, as compared to use of a non-processive polymerizing agent.

Using the present invention, transient state kinetic studies can be efficiently and effectively performed on elongation reactions involving RNAP from human, E. coli, yeast, and bacteriophage T7, and other RNAPs. Many excellent, high-resolution structures of bacteriophage T7 RNAP are available to correlate with kinetic analyses. Many mutations have been constructed in T7 RNAP, and excellent biochemical studies have been done. No transient state kinetic analyses, however, have yet been reported for this important model RNAP. Bacterial RNAP is a useful drug target and a convenient scaffold for constructing mutations. Yeast RNAP II interacts with many elongation factors, and yeast is an optimal eukaryotic system for identification, isolation, and mutation of elongation factors and mutation of RNAP II. The human RNAP II mechanism has been analyzed in detail using transient state methods, but, using the present invention, much more can be done to analyze this enzyme. Similarly, many elongation factors, drugs, and toxins remain to be tested in the human system, and such molecules can be examined using the present invention. No human RNAP II mutants have yet been tested using transient methods, but such mutants can be effectively examined according to the present invention.

An additional biological templated reaction is DNA replication by DNA polymerase (DNAP), another polymerizing agent. DNAPs are not as fully processive as RNAPs. Specifically, DNAPs may disassociate from the DNA template, complicating kinetic analysis. In another embodiment of the present invention, through the formation of several bonds, a DNAP can be covalently tethered to its DNA template and DNA primer without affecting the kinetic behavior of the DNAP. Thus, the DNAP can be rendered completely processive, similar to RNAP. Specifically, DNAPs can be rendered fully processive by preventing disassociation of the DNAP enzyme from the DNA template and DNA primer by DNAP-DNA cross-linking. However, even absent this covalent tethering, DNAP is sufficiently processive to permit kinetic analysis of DNA synthesis according to the present invention.

In a preferred embodiment of the present invention, mitochondrial DNAP γ can be rendered fully processive by flexible cross-linking of mitochondrial DNAP γ to a DNA template, while maintaining reliable and reproducible measurements of elongation kinetics. Other DNAPs that can be evaluated, according to the present invention, include HIV-1 reverse transcriptase and Bacillus Fragment DNAP. Flexible cross-linking agents and covalent DNA tethers (Pierce) can be used to tether the DNAP to the DNA template/primer. The cross-linking agent, however, must be flexible enough to allow the DNAP to move along and elongate the DNA template, while maintaining a strong enough link between the DNAP and the DNA template such that the DNAP and DNA template do not disassociate. Understanding human DNAP γ is important for testing drug candidates for toxicity, particularly antiviral nucleotide analogues, such as AZT.

In another embodiment of the invention, elongation kinetics of polypeptide elongation (i.e., translation) can be studied because ribosomes are fully processive. Thus, an elongation complex can include an immobilized RNA template, a ribosome, and a polypeptide primer. An RNA template including a nonsense codon would cause the ribosome to disassociate from the RNA template, providing an interesting application of the present invention.

Various methods can be used to immobilize or anchor an elongation complex on a membrane for study in accordance with the present invention. Embodiments of the present invention for linking a DNA template to a membrane preferably include attaching the DNA template to the membrane by covalent bonding (e.g., to a Teflon® disc). Alternatively, a biotin-linked elongation complex can be immobilized to streptavadin bound to a membrane. Most preferably, the template (DNA or RNA) is printed on a membrane using microarray DNA immobilization techniques, followed by the addition of the polymerizing agent, and primer formation.

FIG. 2 depicts an elongation complex 10, including a DNA template strand 15, a DNA non-template strand 16, an RNA polymerase 17, and a nascent RNA transcript 18. A biotin group 19 attached to DNA non-template strand 16 of elongation complex 10 immobilizes elongation complex 10 to a streptavadin bead 11 that is bound to a membrane 14.

FIG. 3 depicts an enlarged view of multiple elongation complexes 10 (not shown) immobilized or anchored within sample cells 12 on membrane 14. Each sample cell 12 is an identical replicate print containing at least one femtamole (10⁸) of identical elongation complexes 10. Printing DNA templates 2 on membrane 14 provides a consistent population of elongation complexes 10 in each sample cell 12 and therefore affords a more quantitative experimental method with higher reproducibility. Procedures for immobilizing elongation complexes to a streptavadin agarose bead or immobilized on Ni²+-NTA agarose bead through a histadine tag are known. Komissarova, N. et al., Engineering of Elongation Complexes of Bacterial and Yeast RNA Polymerases, Methods in Enzymology, 371, 233-251.

In a preferred embodiment of the present invention, membrane 14 is a porous, inert material. A porous membrane is preferred in order to permit instantaneous wetting of the bound elongation complexes 10 and rapid transfer of solutions. An inert membrane 14 is preferred so that membrane 14 is not antagonistic to formation or activity of elongation complexes 10 on membrane 14.

The present invention also includes an apparatus 20 for solid-phase kinetic analysis of templated elongation reactions (FIG. 4). Apparatus 20 has a reaction chamber 22, membrane 14, at least two reagent injectors 24 (five are shown in FIG. 4), reagents 26, each in a container, a feed-line 28, a thermal jacket 30, a vessel 32, and a computer controller 34. FIG. 5 is an enlarged view of reaction chamber 22 of FIG. 4 depicting sample cell 12 on membrane 14 located within reaction chamber 22.

In a preferred embodiment of the invention, reagents 26 are delivered under high-pressure flow through reagent injectors 24 to reaction chamber 22, where elongation complexes are immobilized in sample cell 12 located on membrane 14. Reagents 26 are rapidly exchanged with elongation complex 10 so that reaction start-stop times can be as little as one to two milliseconds (or as long as seconds or minutes). The reaction chamber 22 will allow rapid surface mixing (0.1-1.0 millisecond) to ensure sufficiently rapid reagent delivery and mixing. Reagents 26 are delivered from reagent injectors 24 through injector lines 36 to reaction chamber 22. The delivery of reagents 26 to reaction chamber 22 is controlled by computer controller 34. Solid-phase kinetic analysis according to the present invention is a significant improvement over liquid-phase kinetic analysis in that solid-phase kinetic analysis utilizes anchored elongation complexes, and NTPs and reagents are flowed past the anchored elongation complex.

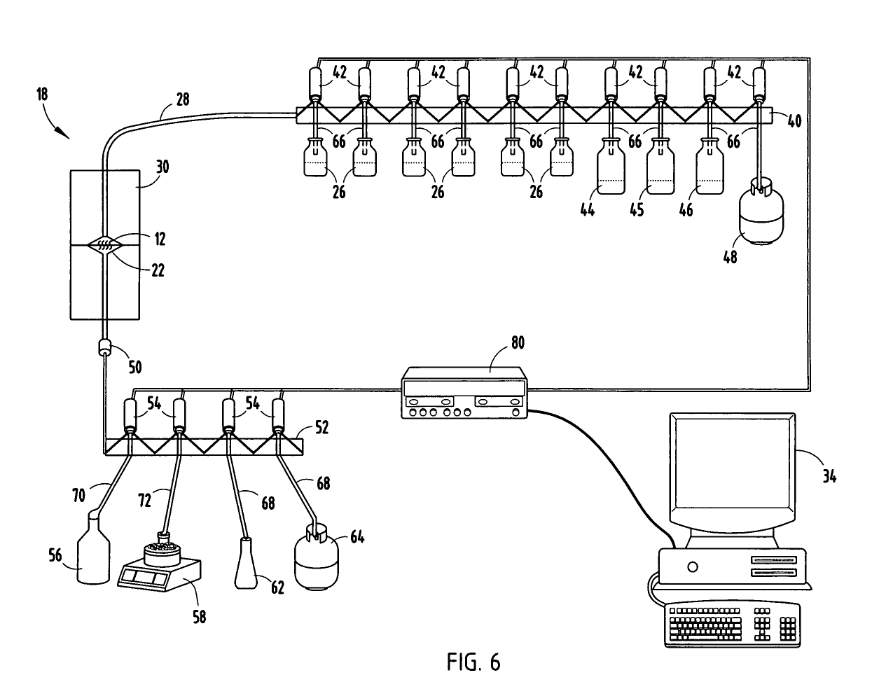

FIG. 6 depicts another apparatus 18 of the present invention in which computer controller 34 controls delivery of reagents 26 to reaction chamber 22 by engaging/disengaging valves (not shown) of a first valve block 40. The valves of first valve block 40 are opened and closed using a first solenoid set 42.

Reaction chamber 22 is adapted to removably receive membrane 14. To maintain a controlled temperature within reaction chamber 22, thermal jacket 30 surrounds reaction chamber 22. Apparatuses 18 and 20 are maintained under pressure because high-pressure flow through reaction chamber 22 allows for the rapid exchange of reagents 26 with elongation complexes 10 with millisecond start-stop times. Preferably, first valve block 40 is a low-volume control, 10-channel valve block (Applied Biosystems) that allows for flexibility in kinetic experimental design.

Reagents 26 include NTPs (ATP, TTP, GTP, CTP, UTP), dNTPs, or amino acylated tRNAs; transcription factors, elongation factors, drugs (including antibiotics), and toxins (e.g., α-amanitin). Various reagents 26 are injected through membrane 14 to which elongation complexes 10 are attached.

Additionally, as shown in FIG. 6, a quench solution 44 and a quench solution 45 are each provided in a container either to stop the elongation reaction or to release the elongated primer from elongation complex 10. Elongation reactions can be quenched either with EDTA, which chelates Mg²⁺, or with HCl, which stops elongation instantly. EDTA is a probe for isomerization of the elongation complex 10, that is, sequestration of the substrate NTP-Mg²⁺ at the active site of the polymerizing agent. HCl is a probe for the chemical step: phosphodiester bond synthesis. As shown in FIG. 6, a buffer solution 46 in a container is provided as the carrying solution for reagents 26.

Also shown in FIG. 6 is a first gas container 48, which mobilizes reagents 26 under pressure into and through reaction chamber 22. Thus, apparatus 18 includes ten-channel first valve block 40 controlling up to eight reagents 26 (seven reagent additions plus a quench/stop solution), which can be reacted in the reaction chamber 22 with elongation complexes 10. Ten-channel first valve block 40 also controls delivery of buffer solution 46 and gas from first gas container 48. Preferably, first gas container 48 contains argon gas. Computer directed reaction protocols operate computer controller 34 to control multiple additions of reagents 26, quench-stop solution 44, buffer solution 46, and argon gas from first gas container 48, with millisecond start-stop timing. When a valve or valves of first valve block 40 is opened by one or more solenoids of first solenoid set 42, one of reagents 26, quench-stop solution 44, buffer solution 46, and/or argon gas from first gas container 48 is delivered through small diameter high-pressure feed-line 28 to reaction chamber 22. A first set of draw lines 66 deliver reagents 26, quench-stop solution 44, buffer solution 46, and gas from first gas container 48 to first valve block 40.

As discussed above with respect to known rapid pipetting devices, NTPs and reagents are reacted with elongation complexes to prime, pulse, chase, and stop the elongation reaction.

Timing of apparatus 18 is continuously monitored by optical sensor 50 (FIG. 6), which measures the rate of flow by sensing exchange of solutions. Because timing is continuously monitored by optical sensor 50, no calibration of apparatus 18 is necessary.

Apparatus 18 also includes a second valve block 52 that is opened and closed using a second solenoid set 54. Computer controller 34 controls delivery of gas from a second gas container 64 (preferably containing argon gas) and reverse-flush solution from a reverse-flush container 62 through second set of draw lines 68 (FIG. 5). Similar to gas that is fed through one of the first set of draw lines 66 from first gas container 48 to first valve block 40, gas is fed from second gas container 64 through one of second set of draw lines 68 to second valve block 52 to maintain reverse pressure in apparatus 18. Specifically, pressure provided by gas from second gas container 64 allows for apparatus 18 to be reverse-flushed by solution contained in reverse-flush container 62.

Control of solenoid switching of first solenoid set 42 and of second solenoid set 54 is directed through switching power supply 80 as directed by computer controller 34.

Apparatus 18 also includes a fraction collector 58. After exposure to reagents, elongated RNA is released from an elongation complex by delivery of quench-stop solution 44 to reaction chamber 22, e.g., HCl is used to release the elongated RNA transcript. In one embodiment of the present invention, the released elongated transcript or elongated polypeptide is automatically delivered to fraction collector 58 through a fraction collection line 72, and the elongated transcript or elongated polypeptide is recovered by fraction collector 58 (FIG. 6). Further, in another embodiment, the fraction collected in fraction collector 58 is manually or automatically transferred to a gel for gel electrophoresis (not shown) or capillary electrophoresis and subsequently to a phosphorimaging or other detection device (not shown). In another embodiment, fraction collector 58 is eliminated and, instead, the released elongated transcript or polypeptide is automatically transferred to an electrophoresis unit for sample analysis (i.e., length determination of elongated transcript or polypeptide).

Apparatus 18 also includes delivery of waste product from reaction chamber 22 through a waste line 70 and to a waste container 56.

Various components of the inventive apparatus are commercially available. For example, valve blocks, solenoids, reaction chambers, and fraction collectors are available from Applied Biosystems.

Anchored elongation complexes are particularly suitable for use with a high-throughput instrument. Specifically, because assays utilizing solid-phase elongation complexes are quantitative and reproducible, these assays can form the scaffold for high-throughput drug development and drug toxicity screens. High-reproducibility can be obtained by automatic or robotic printing of hundreds or thousands of membranes 14 carrying identical populations of immobilized elongation complexes 10 in sample cells 12. Such improved reproducibility of elongation complex printing will ensure that each elongation complex membrane print is essentially identical to every other one. This improvement allows much higher experimental reproducibility, sample to sample, and is obtainable using known instrumentation.

Multiple sample cells 12 can be printed on a single wheel 60 as shown in FIG. 7. In one embodiment, sample cells 12 of wheel 60 are rotated, either manually or automatically, past a single channel apparatus 18 or 20 for solid-phase kinetic analysis, e.g., sample chambers 12 are moved individually into reaction chamber 22 such that elongation complexes 10 in a sample cell 12 are positioned to interact with reagents 26 flowing through membrane 14. Thus, a series of tests can be automated in a series of sample cells 12 on wheel 60. An additional configuration of the present invention includes manually or automatically moving feed-line 28 together with reaction chamber 22 to different sample cells 12.

Another embodiment is a high throughput multiple channel apparatus for solid-phase kinetic analysis (not shown) including multiple reaction chambers 22 in which different sample cells 12 are analyzed. Each channel of this high throughput apparatus is identical (e.g., as described for apparatuses 18 or 20). Such high throughput instruments can be constructed with many channels, e.g., fifty to 200 channels.

There is no specific limit on the number of reagents or channels in the apparatus of the present invention. In a preferred embodiment, the solid-phase kinetic analysis instrument includes 100-plus channels for optimum flexibility in experimental design.

A high-throughput, fully automated solid-phase transient state kinetic instrument may include one or more of the following: automated printing of elongation complexes in a cell on a membrane, automated transfer of the ECs in the cell on the membrane to the reaction chamber, automated reaction protocols, automated transfer to a fraction collector, automated transfer of waste, automated transfer of collected fractions to a gel electrophoresis instrument, and automated transfer of the gel product to a phosphorimaging instrument.

The present invention also includes a method for solid-phase kinetic analysis of templated elongation reactions. One preferred method includes use of an apparatus for solid-phase kinetic analysis, such as apparatuses 18 and 20 (FIGS. 4 and 6). This preferred method further includes preparing elongation complexes 10 in sample cell 12 on membrane 14. As described above, the DNA or RNA template is anchored (e.g., printed) to membrane 14. Preferably, the elongation complex 10 is built by adding a polymerizing agent (such as DNAP, RNAP, or ribosomes) and NTPs (for RNA synthesis), dNTPs (for DNA synthesis), or amino acylated tRNAs (for protein synthesis); and a primer (i.e., a nascent RNA primer, DNA transcript, or polypeptide portion, respectively) is formed. The embodiment further includes the step of moving sample cell 12 containing elongation complexes 10 into reaction chamber 22 of an apparatus for solid-phase kinetic analysis. Next, the operator of the apparatus activates a computer program for operation of the apparatus, according to the desired experimental protocol. In operation, the apparatus flows various reagents 26 past the anchored elongation complexes 10 in reaction chamber 22, thereby elongating elongation complexes 10 and quenching that elongation. In a final quenching step, the elongated primers are released from the elongation complex and recovered. This release/recovery step either can be part of the computerized protocol of the apparatus for solid-phase kinetic analysis or the step can be independently performed subsequent to elongation and quenching of elongation complexes 10.

The preferred method of the present invention also includes analysis of the recovery products (elongated primer fractions). After recovery of elongated primer fractions, the fractions are moved to a gel electrophoresis instrument to observe the elongated transcripts. ³²P can be incorporated into the primer component of elongation complex 10. As such, the elongated primer that is recovered from the solid-phase kinetic analysis apparatus can be readily observed using phosphorimaging equipment. Alternatively, the primer portion of the elongation complex 10 can be fluorescently tagged and the recovered elongated primer observed with fluorescence detection equipment.

Using the method of the present invention, enzymatic activity of various polymerizing agents can be studied. For example, the invention can be used to study human, archaeal, yeast, and bacteriophage RNA polymerases. The method also can be used to study various DNA polymerases, such as human mitochondrial DNAP γ, HIV-1 reverse transcriptase, and Bacillus Fragment DNAP I. Further, the method of the present invention can be used to study ribosomal activity in the translation reaction. Against these polymerizing agents, a researcher can utilize the method of the present invention to examine various elongation factors, mutant elongation factors, drugs, inhibitors of elongation, such as antibiotics (e.g. Microcin and CBR 703 Series), and toxins, such as α-amanitin. Moreover, a researcher can utilize the method of the present invention to examine mutant polymerizing agents.

The above description is considered that of the preferred embodiments only. Modifications of the invention will occur to those skilled in the art and to those who make or use the invention. Therefore, it is understood that the embodiments shown in the drawings and described above are merely for illustrative purposes and not intended to limit the scope of the invention, which is defined by the following claims as interpreted according to the principles of patent law, including the doctrine of equivalents.

What is claimed is:

1. An immobilized elongation reaction system comprising:
a membrane and an elongation complex;
the membrane being compatible with binding the elongation complex; and
the elongation complex being compatible with binding the membrane, the elongation complex including a template, a polymerizing agent, and a primer.

2. The elongation complex of claim 1, wherein the membrane is porous.

3. The elongation complex of claim 1, wherein the polymerizing agent is fully processive.

4. The elongation complex of claim 1, wherein the polymerizing agent is selected from the group consisting of DNA polymerase, RNA polymerase, and a ribosome.

5. The elongation complex of claim 4, wherein the polymerizing agent is a mutated form of one of the group.

6. An apparatus for millisecond kinetic analysis of a templated elongation reaction comprising:
a membrane compatible with anchoring an elongation complex;
a reaction chamber adapted to receive the membrane;
at least two reagents; and
a feed-line interconnecting the reagents and the reaction chamber.

7. The apparatus of claim 6, further including at least one elongation complex linked to the membrane.

8. The apparatus of claim 6, wherein the reagents are selected from the group consisting of NTPs, transcription factors, toxins, drugs, and quench solution.

9. The apparatus of claim 6, further including a computer for controlling the flow of reagents to the reaction chamber.

10. The apparatus of claim 6, further including a valve for controlling delivery of the reagents to the feed-line.

11. The apparatus of claim 10, wherein five valves control delivery of five reagents to the feed-line.

12. The apparatus of claim 6, further including a valve block interconnecting the reagents to the feed-line.

13. The apparatus of claim 7, wherein the elongation complex includes a template, a polymerizing agent, and a primer.

14. The apparatus of claim 13, further including a printer for printing the template on the membrane.

15. The apparatus of claim 7, further including a device for moving the membrane into the reaction chamber.

16. The apparatus of claim 6, further including a fraction collector for collecting the primer.

17. The apparatus of claim 6, further including an apparatus for gel electrophoresis.

18. A method for millisecond kinetic analysis of a templated elongation reaction comprising:
providing an apparatus for millisecond kinetic analysis including a membrane compatible with binding an elongation complex, a reaction chamber, at least two reagents, and a feed-line interconnecting the reagents and the reaction chamber;
binding the elongation complex to the membrane, the elongation complex including a template, a polymerizing agent, and a primer;
moving the membrane into the reaction chamber; and
independently flowing at least two reagents over the elongation complex to permit elongation of the primer and quenching of the elongation complex.

19. The method of claim 18, wherein binding further includes printing at least one template on the membrane.

20. The method of claim 18, wherein independently flowing further includes providing a computer for controlling a flow of the reagents to the reaction chamber.

21. The method of claim 18, further including flowing reagent past the elongation complex to remove the primer from the elongation complex.

22. The method of claim 21, further including collecting the primer in a fraction collector.

23. The method of claim 22, further including moving the primer from the fraction collector to an electrophoresis apparatus.
